SEQUENCE LISTING (69 sequences)

SEQ ID NO: 1 - DNA, 21 bp, Homo sapiens
  gene (1)..(21)
aacctttcttttctcttccaa

SEQ ID NO: 2 - DNA, 20 bp, Homo sapiens
  gene (1)..(20)
ccaaaagaagaaagaaggtc

SEQ ID NO: 3 - DNA, 19 bp, Homo sapiens
  gene (1)..(19)
ttaaagagaggagaaggtg

SEQ ID NO: 4 - DNA, 25 bp, Homo sapiens
  gene (1)..(25)
gtgagagagaaagagaaagagagtg

SEQ ID NO: 5 - DNA, 21 bp, Homo sapiens
  Gene (1)..(21)
tattctttccttttcttttac

SEQ ID NO: 6 - DNA, 19 bp, Homo sapiens
  Gene (1)..(19)
ttgagagggaaagggaata

SEQ ID NO: 7 - DNA, 21 bp, Homo sapiens
  Gene (1)..(21)
atgggagaggaggagaagatg

SEQ ID NO: 8 - DNA, 19 bp, Homo sapiens
  Gene (1)..(19)
agttccttttcctttccat

SEQ ID NO: 9 - DNA, 21 bp, Homo sapiens
  Gene (1)..(21)
gatccttcctttctcccctgt

SEQ ID NO: 10 - DNA, 35 bp, Homo sapiens
  Gene (1)..(35)
ctgaaggaaggaaggagggaggaaggaaggaagca

SEQ ID NO: 11 - DNA, 22 bp, Homo sapiens
  Gene (1)..(22)
gtagagggaagggagaaaggtg

SEQ ID NO: 12 - DNA, 19 bp, Homo sapiens
  Gene (1)..(19)
tcaaaagaaggagaagatg

SEQ ID NO: 13 - DNA, 21 bp, Homo sapiens
  Gene (1)..(21)
tgtcttcctttcttcctccat

SEQ ID NO: 14 - DNA, 23 bp, Homo sapiens
  Gene (1)..(23)
tgtctctctctctcctccccccc

SEQ ID NO: 15 - DNA, 30 bp, Homo sapiens
  Gene (1)..(30)
cacccctctctctccttctctcttttctgt

SEQ ID NO: 16 - DNA, 19 bp, Homo sapiens
  Gene (1)..(19)
tgctcccctccctcctcac

SEQ ID NO: 17 - DNA, 20 bp, Homo sapiens
  Gene (1)..(20)
cctcctcttctctccctccc

SEQ ID NO: 18 - DNA, 16 bp, Homo sapiens
  Gene (1)..(16)
tcttccttttctctct

SEQ ID NO: 19 - DNA, 15 bp, Homo sapiens
  Gene (1)..(15)
ctttccctccctctt

SEQ ID NO: 20 - DNA, 15 bp, Homo sapiens
  Gene (1)..(15)
ccttcttctttcccc

SEQ ID NO: 21 - DNA, 16 bp, Homo sapiens
  Gene (1)..(16)
cctcctcttttctcct

SEQ ID NO: 22 - DNA, 20 bp, Homo sapiens
  Gene (1)..(20)
ttccttccttccctctttcc

SEQ ID NO: 23 - DNA, 24 bp, Homo sapiens
  Gene (1)..(24)
ctcttttcttttcttttctttctt

SEQ ID NO: 24 - DNA, 16 bp, Homo sapiens
  Gene (1)..(16)
ctccttcctttctctc

SEQ ID NO: 25 - DNA, 18 bp, Homo sapiens
  Gene (1)..(18)
ttctctttcttctcttct

SEQ ID NO: 26 - DNA, 15 bp, Homo sapiens
  Gene (1)..(15)
tttctctttcctcct

SEQ ID NO: 27 - DNA, 17 bp, Homo sapiens
  Gene (1)..(17)
tctttccctttccctcc

SEQ ID NO: 28 - DNA, 17 bp, Homo sapiens
  Gene (1)..(17)
cctcccttcccccttcc

SEQ ID NO: 29 - DNA, 17 bp, Homo sapiens
  Gene (1)..(17)
ctccttttcccttccct

SEQ ID NO: 30 - DNA, 19 bp, Homo sapiens
  Gene (1)..(19)
ccctttccctttctctccc

SEQ ID NO: 31 - DNA, 18 bp, Homo sapiens
  Gene (1)..(18)
cccccttcctcccctttc

SEQ ID NO: 32 - DNA, 17 bp, Homo sapiens
  Gene (1)..(17)
tctctctccctcccctc

SEQ ID NO: 33 - DNA, 22 bp, Homo sapiens
  Gene (1)..(22)
tcttctctcctcttctctcctc

SEQ ID NO: 34 - DNA, 18 bp, Homo sapiens
  Gene (1)..(18)
cttcctcccttctcctcc

SEQ ID NO: 35 - DNA, 18 bp, Homo sapiens
  Gene (1)..(18)
ccctcccccctccctccc

SEQ ID NO: 36 - DNA, 16 bp, Homo sapiens
  Gene (1)..(16)
ccccttcctcctccct

SEQ ID NO: 37 - DNA, 16 bp, Homo sapiens
  Gene (1)..(16)
cctccttctcccctct

SEQ ID NO: 38 - DNA, 21 bp, Homo sapiens
  Gene (1)..(21)
cctctctccctttttcttctt

SEQ ID NO: 39 - DNA, 17 bp, Homo sapiens
  Gene (1)..(17)
agaagggaagggaggga

SEQ ID NO: 40 - DNA, 18 bp, Homo sapiens
  Gene (1)..(18)
agaggggaagggaggagg

SEQ ID NO: 41 - DNA, 16 bp, Homo sapiens
  Gene (1)..(16)
ctctctttctctcccc

SEQ ID NO: 42 - DNA, 18 bp, Homo sapiens
  Gene (1)..(18)
cctctctcctctccttcc

SEQ ID NO: 43 - DNA, 19 bp, Homo sapiens
  Gene (1)..(19)
aggagggaggaagagaggg

SEQ ID NO: 44 - DNA, 17 bp, Homo sapiens
  Gene (1)..(17)
ccccctctccttctcct

SEQ ID NO: 45 - DNA, 22 bp, Homo sapiens
  Gene (1)..(22)
ggggaggagagaaaagaaggag

SEQ ID NO: 46 - DNA, 17 bp, Homo sapiens
  Gene (1)..(17)
cctccctttcctctccc

SEQ ID NO: 47 - DNA, 22 bp, Homo sapiens
  Gene (1)..(22)
aggagaggagagagagggaaga

SEQ ID NO: 48 - DNA, 22 bp, Homo sapiens
  Gene (1)..(22)
ccttccttcccccctctcccct

SEQ ID NO: 49 - DNA, 21 bp, Homo sapiens
  Gene (1)..(21)
gggggaggggaagaaggaggg

SEQ ID NO: 50 - DNA, 16 bp, Homo sapiens
  Gene (1)..(16)
ttcctttctcctcctc

SEQ ID NO: 51 - DNA, 17 bp, Homo sapiens
  Gene (1)..(17)
ctcccttttctcttctt

SEQ ID NO: 52 - DNA, 19 bp, Homo sapiens
  Gene (1)..(19)
ctttctccccttctccctc

SEQ ID NO: 53 - DNA, 16 bp, Homo sapiens
  Gene (1)..(16)
aaggagaaaaggagga

SEQ ID NO: 54 - DNA, 16 bp, Homo sapiens
  Gene (1)..(16)
gaggggagaagggagg

SEQ ID NO: 55 - DNA, 15 bp, Homo sapiens
  Gene (1)..(15)
cctcctctctctccc

SEQ ID NO: 56 - DNA, 19 bp, Homo sapiens
  Gene (1)..(19)
gggaaggagaagaggaagg

SEQ ID NO: 57 - DNA, 17 bp, Homo sapiens
  Gene (1)..(17)
cccctcctccttcctct

SEQ ID NO: 58 - DNA, 15 bp, Homo sapiens
  Gene (1)..(15)
agaggaagagaagaa

SEQ ID NO: 59 - DNA, 22 bp, Homo sapiens
  Gene (1)..(22)
ccttctttcctctctccttccc

SEQ ID NO: 60 - DNA, 16 bp, Homo sapiens
  Gene (1)..(16)
ccctttctcctccccc

SEQ ID NO: 61 - DNA, 28 bp, Homo sapiens
  Gene (1)..(28)
tctcttttcctttctcttccccctcctc

SEQ ID NO: 62 - DNA, 15 bp, Homo sapiens
  Gene (1)..(15)
aaaggaagagaagaa

SEQ ID NO: 63 - DNA, 16 bp, Homo sapiens
  Gene (1)..(16)
cccctttcccttccct

SEQ ID NO: 64 - DNA, 19 bp, Homo sapiens
  Gene (1)..(19)
ccccccttcctctcctctt

SEQ ID NO: 65 - DNA, 25 bp, Homo sapiens
  Gene (1)..(25)
gggggggagggaagagagaaagaga

SEQ ID NO: 66 - DNA, 16 bp, Homo sapiens
  Gene (1)..(16)
gaagagagggagaaag

SEQ ID NO: 67 - DNA, 17 bp, Homo sapiens
  Gene (1)..(17)
gagaaggaaggagagag

SEQ ID NO: 68 - DNA, 16 bp, Homo sapiens
  Gene (1)..(16)
cctctttcctcctctc

SEQ ID NO: 69 - DNA, 17 bp, Homo sapiens
  Gene (1)..(17)
tccttccctccccctct

(19) United States
(12) Patent Application Publication
(10) Pub. No.: US 2009/0130686 A1
(43) Pub. Date: May 21, 2009

(54) Method for the Microscopic Localization of a Selected, Intracellular DNA Segment with a Known Nucleotide Sequence

(75) Inventors: Michael Hausmann, Ludwigshafen (DE); Christoph Cremer, Heidelberg (DE)

Correspondence Address: JOYCE VON NATZMER;PEQUIGNOT + MYERS LLC, 200 Madison Avenue, Suite 1901, New York, NY, 10016, US

(73) Assignee: RUPRECHT-KARLS-UNIVERSITAET- HEIDELBERG, Heidelberg (DE)

(21) Appl. No.: 12/294,381

(22) PCT Filed: Feb. 21, 2007
(86) PCT No.: PCT/EP07/01467

§ 371 Date: Jan. 28, 2009

(30) Foreign Application Priority Data
Mar. 25, 2006 (EP) 06006213.0

(51) Int. Cl. C12Q 1/68; C07H 21/04

(52) U.S. Cl. 435/6; 536/24.3; 977/774

(57) ABSTRACT

The method for the microscopic localization in situ of a selected intracellular native genome segment with a known nucleotide sequence is characterized by the nature and the sequence of the following measures: (1.) The target DNA is analyzed, via genome databases, for partial sequences which constitute a unique pattern within the genome. (2.) Single-stranded probe sequences are provided which are identical to these partial sequences or complementary thereto, and which are suitable for hybridizing with the single strands of these subsequences via a Watson-Crick binding. (3.) The probe sequences are coupled with marker molecules, where all units of probe sequence and marker molecule(s) have the same binding behavior or the same melting point as the single strand of the target DNA complementary thereto. (4.) The probe sequences are introduced into the cell and combined with the target DNA so that they hybridize to the corresponding partial sequences, of the target DNA, which are temporarily present as two single strands. (5.) The marker signals emitted are detected, and (6.) the locus of the target DNA on the genome is identified on the basis of the presence and/or intensity and/or the simultaneous occurrence of different marker signals.

The invention relates to a method for the microscopic analysis of the localization of a selected intracellular native genome segment having a known nucleotide sequence—“target DNA” hereinafter—in particular of a gene within one (or on one) chromosome, in situ.

The specific labeling of DNA regions (“target DNAs”) in cell nuclei and on chromosomes with the aid of specific DNA, RNA or PNA probes is known in the art. The probes are single-stranded molecule chains which are prepared by molecular biological amplification methods such as, for example, cloning or polymerase chain reaction, and which have corresponding complementary target sequences in a genome. Such probes are normally prepared from BAC, cosmid or YAC clones which are suitably combined, or which are adapted by molecular biological methods to the target sequence. These BAC, cosmid or YAC probes establish the size and the site of the labeling. The length of these probes or of the complementary sequences often does not agree exactly with, but exceeds, the desired DNA region which is to be labeled, especially in the case of small genes, frequently by a multiple of this target DNA. One example thereof is the Her2/neu gene, which is relevant to tumor diagnosis and therapy, on chromosome 17. This gene has a length of about 44 kb. All the BAC clones generally available for labeling this gene have a length of the order of about 100 kb. When the Her2/neu gene is labeled by fluorescence in situ hybridization there is thus frequently additional labeling of adjacent genes such as, for example, of the gene Grb7. A differential microscopic analysis of the individual changes in the individual Her2/neu gene is thus impossible without complex molecular biological modifications of the probes.

A similar problem exists in the experimental determination of the position of breakpoints in chromosomes which are involved in translocations. Such breakpoints can indeed be delimited by overlapping BAC clones to a region of less than 10 kb. However, a more accurate localization then requires sequencing methods or PCR methods which do not experimentally provide any information about the individual cell and, in addition, are relatively labor-intensive. It is desirable here to reduce this labor-intensiveness through the labeling regions being smaller and more accurately definable.

Fluorescence labeling methods for genome regions in cell nuclei and on chromosomes with specific DNA, RNA, PNA probes are normally referred to in the literature as fluorescence in situ hybridization (FISH). For the labeling, reporter molecules having for example a high affinity for corresponding fluorochrome complexes are incorporated in these probes. However, certain fluorochrome complexes can also be incorporated directly into the probe molecules, where appropriate via a linker of suitable length. The range of colors of the fluorochromes available extends over the visible spectrum into the infrared. Besides the emission spectrum, it is also possible to utilize the lifetime of the fluorescence emission as parameter. The properties of absorption spectrum, emission spectrum and fluorescence lifetime are referred to as spectral signature. Besides the fluorescent marker molecules, further signatures which enable specific identification of the labeled genome region are known, e.g. those employed in atomic force microscopy (gold, silver molecules) or in magnetic resonance imaging (paramagnetic nanoparticles).

Detection of the labeled target genome regions normally takes place by far-field microscopy (e.g. epifluorescence microscopy, confocal laser scanning microscopy, wave field/SMI microscopy, fluorescence correlation microscopy, with or without supplementary axial tomographic techniques, 4Pi microscopy etc.), by near-field microscopy (AFM, SNOM etc.), by magnetic resonance methods or by other methods for accurate determination of the location of the markers.

Most of the hybridization protocols employed to date in FISH assume that, as precondition for this process, not only the probe but also the target DNA, which is frequently in double-stranded form in nature, must be converted completely into a single-stranded form (denaturation). Either chemical treatment methods (e.g. using an appropriately high concentration of the solvent formamide or other chaotropic substances), enzymatic and/or thermal treatment methods (e.g. heating to temperatures above 70° C.) have been prescribed or proposed for the denaturation. The renaturation which is subsequently necessarily brought about then leads to the formation of hybrid double strands of probe and target DNA (standard FISH).

It is further known that single-stranded probes in which the sugar-phosphate backbone has been replaced by a polyamide chain (so-called PNA probes) can combine in a sequence-specific manner with single- or double-stranded DNA to give a new two- or three-stranded formation (PNA-DNA). This knowledge has been implemented experimentally to visualize certain repetitive sequences by FISH (PNA-FISH). Owing to the elimination, brought about by the use of PNA, of the electrostatic repulsive forces between target DNA and probe PNA, this method can be carried out with substantially any base sequences and even in the presence of high concentrations of formamide; however, it has the disadvantage of considerable labor in the synthesis of the PNA probes.

In relation to the question about the mechanism of hybridization, it has to date been assumed in the art that the single-stranded DNA segments present naturally in each genome are insufficiently numerous for in situ hybridization. On the other hand, the publication by Winkler et al. (Journal of Microscopy, 2003) describes specific hybridization of certain DNA probes onto target chromosomal sequences even if no denaturation of the target has been carried out. These results are supported by Dobrucki J. and Darzynkiewicz Z. (2001) Micron, vol. 32, pp. 645-652.

In addition, DE 198 06 962, and Hausmann et al. (2003), Biotechniques, volume 35, pp. 564-577, and Schwarz-Finsterle et al. (2006), Cell Biology International, volume 29, pp. 1038-1046, describe an in situ hybridization method which dispenses with denaturation and hybridizes single-stranded homopurine or homopyrimidine sequences as probes onto the double-stranded target DNA via Hoogsteen linkages. The single-stranded homopurine or homopyrimidine probes bind to the bases of the double-stranded duplex nucleic acid sequences and thus form a three-stranded triple helix. This creates the preconditions for FISH on living or vital cells since these probes can be introduced into such cells by microinjection methods and then form specific adducts with the complementary segments of the duplex DNA without further treatment of the cells being necessary. However, such triple helix-forming sequences represent only about 2% of the genome, i.e. not every desired genome region can be specifically and completely encompassed by this method.

To date, in vivo labeling methods have been disclosed alternatively for: (i) nucleoli with the aid of fluorescence in situ hybridization of RNA probes (RNA-FISH); (ii) centromer regions with a centromere-specific protein which was visualized with the aid of a coupled green fluorescent protein (GFP); (iii) certain homogeneously staining regions (HSR) with a Lac operon-specific GFP-coupled protein; and (iv) whole chromosome territories utilizing replication mechanisms.

U.S. Pat. No. 5,176,996 moreover discloses that it is possible to synthesize defined single-stranded probe oligonucleotides which bind specifically to selected double-stranded DNA sequences, resulting in a triple strand, so that gene functions such as protein synthesis are blocked. The described probe oligonucleotides normally have a length of more than 20 nucleotides and carry no marker molecules.

WO 95/03428 describes a method for the in situ analysis of the localization of a specific nucleic acid with the aid of modified single-stranded probe oligonucleotides which are complementary to the target DNA sequence. The probe sequence, a single oligonucleotide sequence, is modified in order to make diffusion into the cell possible. WO 95/03428 does not, however, contain any information about determination of the probe sequence and selection and establishment of selecting probe parameters.

However, none of the disclosed FISH methods or alternative in vivo labeling methods allows nucleotide-accurate in situ labeling of any desired and, where appropriate, also very small genome regions practically (virtually) without changing the natural functional organization specifically in vital cells.

However, there is a need both in fundamental research and in particular in medical diagnosis for a method with which nucleotide-accurate visualization even of small genome regions is possible and, in the ideal case, additionally mutations, amplifications or deletions thereof. It should be possible in this connection for the genome material to be labeled and analyzed if possible without, or with only minor, pretreatment in order to avoid damage to the natural structures. This means that the genome material and thus consequently also the relevant cell(s) are maintained and must remain in vivo or in a (vital) state approaching the in vivo state. However, none of the in situ hybridization methods which have been disclosed to date and with which it is possible to speak of a vitally preserved target genome permits any FISH of any desired specific, small genome segments such as, for example, individual genes or tumor-relevant genome loci.

The present invention therefore addresses the problem of providing a method with which to label, nucleotide-accurately and without, or at the most extremely small, alterations in the native structural and functional organization, any desired genome regions which in some circumstances are also very small and known in terms of their nucleotide sequence, so that localization of this genome region in or on the chromosome or genome is possible with the aid of microscopic techniques, and thus also subsequent procedures for which knowledge of the accurate localization of a gene is a prerequisite.

One solution to this problem consists of a method of the type mentioned at the outset, which is characterized by the nature and sequence of the following measures, namely in that (1.) the target DNA is analyzed on the basis of (known) genome databases in relation to (a) a single or (b) a plurality of identical or different partial sequences, where these partial sequences or the combination of the plurality of identical or different partial sequences represent a unique pattern within the genome, meaning that there is no occurrence in the remaining genome of this specific arrangement in which it is or they are present within the target DNA, in that (2.) single-stranded probe sequences are provided which coincide with or are complementary to the partial sequence(s) of the target DNA, and which are suitable for hybridization via Watson-Crick binding to the partial sequence(s) which is/are in the form of two DNA single strands of the target DNA—naturally at least temporarily (for a time, transiently), e.g. during cell division—, in that (3.) these probe sequences are coupled to identical and/or different marker molecules which can detected by microscopy or spectroscopy or by magnetic resonance, where in the case (1b) of a combination of a plurality of partial sequences or probe sequences and coupling thereof to different marker molecule(s) each unit of probe sequence and marker molecule(s) displays virtually the same binding behavior or the same melting point with the single strand, complementary thereto, of the target DNA as each of the other units (meaning in other words: all the units of probe sequence and marker molecule(s) have essentially the same binding behavior, namely the same binding energy and/or binding kinetics, and/or the same melting point with the single strand of the target DNA segment), in that (4.) these probe sequence(s) are introduced into the cell and brought together with the target DNA by known methods in such a way that they hybridize with the corresponding partial sequence(s), which is/are temporarily (transiently, for a time) in the form of two DNA single strands of the target DNA, of the target DNA, in that (5.) the emitted marker signals are detected, and in that (6.) the location of the target DNA on the genome, in particular the chromosome, is identified on the basis of the presence and/or the intensity and/or the simultaneous occurrence of different marker signals.

(Human) DNA is naturally temporarily (transiently, for a time) in the form of two DNA single strands for example during cell division, especially during DNA duplication. Such a situation of “naturally temporarily in the form of single strands” can also be induced or stimulated by the skilled person if required by stimulating or inducing for example cell division of the relevant cells.

The expression “binding behavior” stands here and hereinafter for the combination “binding energy and binding kinetics and numerical ratio of purines to pyrimidines”.

The advantage of this method is that there is accurately focused labeling of any desired genome regions and not, as with the FISH methods customary to date, the probes are determined by the size of the molecular biologically prepared BAC, cosmid or YAC clones and their binding sites, so that in particular genome regions of a few kb are hit only inaccurately or far too comprehensively.

Since the probes can be synthesized with the same sequence and additionally with different orientation depending on the desired FISH method (standard FISH with denaturation of the target DNA, FISH without denaturation, FISH with DNA single strands or double strands etc.), this method can be adapted with a high degree of flexibility to the particular target material and its quality.

The method is preferably carried out in vivo or vitally (i.e. close to the in vivo state) in order to ensure a maximum level of accuracy in the localization of the gene or gene segment to be visualized.

The partial sequence and thus also the probe sequences should have a length of at least 8, preferably 10 to 40 nucleotides.

Proposed marker molecules are in particular fluorescent dyes, immunohistochemical marker molecules, fluorescent or luminescent nanoparticles, especially nanocrystals (quantum dots), and magnetic nanoparticles, it also being possible to employ combinations of different marker molecules of these types.

A development of the method which can be employed advantageously in particular in tumor diagnosis makes it possible to detect point mutations and/or genome breakpoints and/or equivalent sequence defects (microdeletions), and is characterized in that in step (1) of the basic method the target DNA is analyzed for a plurality of (identical or different) partial sequences t₁-tₙ, in that in step (3) the probe sequences S₁-Sₙ are coupled to different marker molecules which can be detected by microscopy or spectroscopy or by magnetic resonance, and in that in step (6) the absence of the marker signals of one or more probe sequences Sₓ with simultaneous presence of the marker signals of probe sequences Sₙ-Sₓ indicates the presence of a point mutation or of a genome breakpoint or of an equivalent sequence defect.

It is possible in particular for this developed method to construct the probe sequences in such a way that they no longer bind to the target DNA as soon as only one of their bases is not complementary to the target sequence. This makes it possible to discover a mutation on the basis of the absence of the signal of a particular probe sequence in a probe mixture. Deletions and, in particular, microdeletions can also be discovered in this way.

Breakpoints within the target DNA can likewise be identified and delimited with this developed method. They are indicated by the fact that the different probe sequences of a provided probe set do not all bind to a genome locus, but are detected on two or more genome loci at a distance from one another.

The probes and/or probe sets are introduced into the target cells by standard methods of biochemistry, molecular biology or cytogenetics. Preference is given to diffusion methods, methods of microinjection, methods of electroporation and methods of vesicle-activated membrane transfection.

To solve the stated problem, special probe sets are additionally proposed for in situ hybridization and microscopic analysis which comprises a single or a plurality of probe type(s) S₁-Sₙ, where each probe type Sᵢ comprises/consists of an oligonucleotide or a molecule equivalent thereto (e.g. a PNA oligomer) which is complementary or identical to a single-stranded partial sequence tᵢ of an intracellular, double-stranded genome segment having a known nucleotide sequence—“target DNA” hereinafter—, in particular of a gene within or on a chromosome. This probe set is characterized in that the single or the plurality of partial sequences in combination represent a unique pattern within the genome [i.e.: they do not occur in the remaining genome, in particular chromosome, in the specific arrangement in which they are present within the target DNA], in that the probes are suitable for hybridization via Watson-Crick binding to the partial sequence(s) which is/are in the form of two DNA single strands of the target DNA (as is the case naturally, i.e. in vivo or in the native state, at least temporarily or transiently or for a time), and in that the probes are coupled to identical and/or different marker molecules which can be detected by microscopy or spectroscopy or by magnetic resonance, where in the case of the combination of a plurality of probe types and their coupling to different marker molecule(s) each unit of probe type and marker molecule(s) shows virtually the same binding behavior or the same melting point with the single strand, complementary thereto, of the target DNA as each of the other units [meaning in other words: all units of probe type and marker molecule(s) have essentially the same binding energy or the same melting point with the single strand of the DNA target segment].

The oligonucleotides of this probe set should have at least 8 nucleotides, preferably 10-40 nucleotides. A corresponding statement applies where appropriate to oligonucleotide-equivalent molecules.

Marker molecules proposed for the probe sets of the invention are in particular fluorescent dyes, immunohistochemical marker molecules, fluorescent or luminescent nanoparticles, in particular nanocrystals (quantum dots), and magnetic nanoparticles, it also being possible to employ combinations of different marker molecules of such types.

The method of the invention and the probe sequences and probe sets of the invention are intended for focused labeling of genome regions in fundamental research, applied research and medical diagnosis. A good signal/background ratio in the detection is necessary in particular for medical diagnosis. This is achieved with the method of the invention, especially with the probe sequences and labeling strategies of the invention (nanocrystals, fluorescence quenching etc.).

The following microscopic methods are equally suitable in particular for the microscopic detection and imaging of the marker signals: the fluorescence microscopy, especially optical near-field microscopy and optical far-field microscopy such as, for example, epifluorescence microscopy, confocal laser scanning microscopy, wave-field microscopy, spatially modulated illumination (SMI) microscopy, 4Pi microscopy and other high-resolution microscopic techniques.

Suitable probe sequences and probe sets of the invention can also be detected with the aid of flow cytometry and slit scan flow fluorometry, and sensor technology and magnetic resonance tomography.

Because of the facts that (I) accurate labeling of extremely small target DNAs is possible with the method of the invention, and that (II) it is moreover possible to dispense with pretreatment of the cell material, in particular a chemical, enzymatic and/or thermal denaturation, because the probe sequences and probe sets of the invention hybridize in vivo and in situ (because each double-stranded DNA is naturally at least temporarily—meaning transiently or for a time—and segmentally “open” in the form of two single strands), this method makes it possible, for example with the aid of SMI microscopy, to estimate compaction of the relevant genome region and thus to detect changes during cell function or tumorigenesis in the DNA structure.

The method of the invention very considerably simplifies the handling of patient's material as target in diagnosis. In particular, it is unnecessary or scarcely necessary to alter physiologically relevant conditions as are generally present when obtaining patient's material. This is a crucial advantage also in relation to the mild treatment of the cell material to be analyzed in order to preserve relevant structural information.

The method of the invention and the probes of the invention opens up for general and clinical research and medical diagnosis in particular also the advantageous possibility of specific in vivo labeling of genome structures in the cell nucleus for analysis of genome loci by magnetic resonance tomography methods.

Owing to the research on the DNA sequence of the human and other genomes, comprehensive DNA sequence libraries are currently available on appropriate computers. The method of the invention and its development including the probes of the invention can be employed with all (these) genomes with known nucleotide sequence for localizations of genes or for detection of gene alterations (breaks, deletions, mutations etc.). It is thus also possible to produce specific labelings for species for which only a few, or no, DNA banks with appropriate BAC clones are available at present. This advantage is of considerable, also clinical, interest, e.g. in relation to simplified labeling of DNA sequences of particular pathogens.

The invention is explained in more detail below by means of figures and examples.

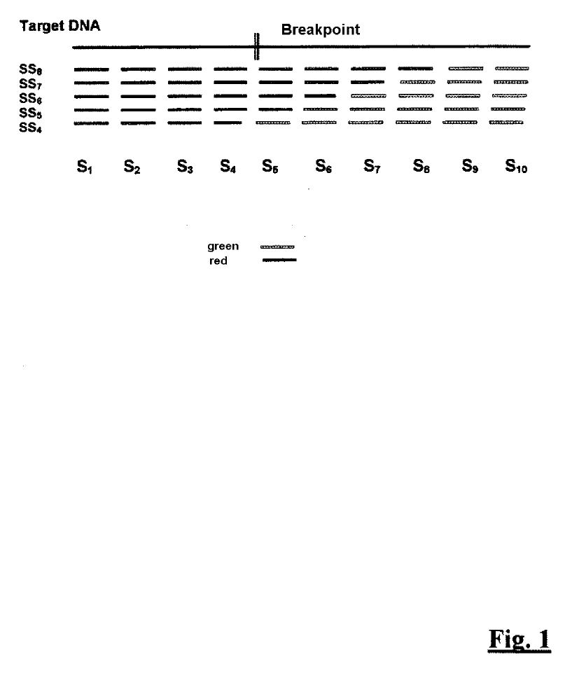

FIG. 1 shows a diagrammatic representation of the five
probe set variants SS₄ to SS₈ according to example 6, depicting only the first 10 probes S₁ to S₁₀ of each probe set;
probe set variant SS₄ comprises probes S₁ to S₄ with red labeling (dark bar) and probes S₅ to S₁₀ with green labeling (pale bar), probe set variant SS₅ comprises probes S₁ to S₅ with red labeling (dark bar) and probes S₆ to S₁₀ with green labeling (pale bar), probe set variant SS₆ comprises probes S₁ to S₆ with red labeling (dark bar) and probes S₇ to S₁₀ with green labeling (pale bar), etc.;
probe set variant SS₄ provides two monochrome marker signals since the breakpoint is located in the region between the red-labeled (dark bar) single probe S₄ and the green-labeled (pale bar) single probe S₅.

### EXAMPLE 1

### Preparation of Probe Sets of the Invention

Probe sets of the invention are prepared by selecting for example in a DNA sequence library a DNA sequence which is to be labeled, is continuous apart from minor gaps and has total length L, the “target DNA”. L means in this connection a linear geometric length of a DNA thread of the complete base sequence (1 kbp can be estimated to be about L=350 nm). This length L may in the untreated cell nucleus correspond to a genome region or chromosomal subregion which is to be labeled and whose average diameter d_{T} is less than or equal to the half width of the chief maximum of the effective pixel function (or resolution-equivalent quantity of the detection system) of the detection system used for the subsequent analysis of the target DNA or cell.

The target DNA of length L is screened for partial sequences with suitable binding behavior (binding energy and/or binding kinetics) and/or suitable melting point. Such partial sequences may be for example 15 mers (=oligonucleotides consisting of 15 nucleotides) which—in the simplest case—have a fixed ratio of purine and pyrimidine bases. These partial sequences are compared with the remainder of the genome, and only those which do not occur in combination (including repetition) in the remainder of the genome within any desired genome segments of a length L are selected. The partial sequences are then transcribed into corresponding complementary probe sequences and synthesized as PNA and/or DNA and/or other equivalent sequence-specific probes. The abovementioned marker molecules and nanocrystals are subsequently linked where appropriate via suitable linker molecules or molecule groups to these probes.

The mixing ratio of the various individual probes involved in the probe mixture may be for example 1:1; other mixing ratios are expressly permitted. The probes may moreover carry the same signature or different signatures.

### EXAMPLE 2

### Labeling of the Probes (Sequences Thereof)

The individual probes/probe sequences may be labeled differently for the detection:

a) Fluorescent molecules can be attached, where appropriate via suitable linker molecules, to the 3′ and/or 5′ end of the probe sequence. These fluorescent molecules may show the same spectral signature for all the different probe sequences of a probe mixture. In this case, the localization and completeness of the labeling can be detected via the intensity. The different probe sequences of a probe set may, however, also have different spectral signatures. In this case, the spectral composition, besides the intensity, of the detected probe set is also a suitable parameter for the localization and completeness of the labeling.

b) The fluorescent molecules may just as well be incorporated into the individual probe sequences not on the margins. The same as described in (a) applies to the detection in this case.

c) The fluorescent molecules may, however, also have their fluorescence impeded in the unbound state by quencher molecules. These molecules may be coupled directly to one end of the probe sequence as for example in the case of PNA probes. Owing to the flexibility of the PNA, quenching always takes place until the specific binding of the probe to the target DNA has taken place. In the case of DNA, it is possible to suppress the fluorescence by addition of nucleotides or molecules in a stem-loop structure of the probe sequence (so-called smart probes or hairpin probes). The stem is opened only when there is specific binding of the loop, and the fluorescence is emitted.

d) Instead of fluorescent molecules it is possible to incorporate magnetic marker molecules, marker molecules with a high affinity for dye complexes, e.g. steroids or haptens, or molecules with a high atomic interaction with atomic force microscopy tips, into the probe sequences or for them to be coupled thereto. In the case of marker molecules with a high affinity for dye complexes, the statements in (a), (b) and (c) apply analogously in relation to the dye complexes to be bound.

e) The labeling can just as well take place by means of fluorescent/luminescent and/or magnetic nanoparticles (quantum dots).

In principle, all the probes are labeled in such a way that their marker signal stands out significantly by comparison with a nonspecific background labeling in the event of binding to the target DNA.

### EXAMPLE 3

### Localization of a Gene X in Genome G

Gene X is the target DNA “T”. An initial and final nucleotide is established for this target DNA in order for the region located in between to be specifically labeled subsequently.

A database search is carried out in a genome database, and at least one DNA sequence is determined, preferably a combination of DNA sequences—the so-called “partial sequences”—, which are colocalized, namely occur together, within the target DNA (i.e. in the region or over the length L of the target DNA), and which do not occur in this combination in the remaining genome G in any genome segment of length L.

Single-stranded probe sequences consistent with or complementary to these partial sequences and able to hybridize via Watson-Crick bindings to the matching DNA single strand of these partial sequences—as soon as this partial sequence is in the form of two single strands, or which repeatedly occurs naturally from time to time (=temporarily, transiently), are prepared.

The probe sequences or partial sequences are selected in this connection according to the following criteria:
A sufficient number of probe sequences must bind within (in the region or over the length of) the target DNA for there to be at least one probe within the resolution limits of the detecting system intended to be used.
If the target DNA can be defined by a single probe sequence, because the relevant (complementary) partial sequence is unique in the relevant genome, it is sufficient to provide this single probe sequence.
If the target DNA cannot be defined by a single partial sequence and thus probe sequence, it is necessary to provide a combination of probe sequences, also called “probe mixture” hereinafter. The probe sequences of this combination or of this probe set are selected in each case such that the individual probes are all colocalized (=occur together) together only in the region of the target DNA, but not in the remaining genome.
In order to ensure uniform binding of the individual probes of a probe set to the target DNA, the various probe sequences should be selected such that they have the same binding behavior or the same melting point with the DNA strand. These parameters may also be varied within preset limits.

The chosen probe sequences are subsequently coupled to marker molecules. Particularly suitable labels are the following:

(a) The probes are coupled to fluorescent dyes whose intensity and/or spectral signature (colors and/or fluorescence life time) serve(s) as detection parameter.
(b) The probes are coupled to fluorescent/luminescent nanocrystals (quantum dots).
(c) Probe constructs which, owing to fluorescence-quenching molecules or because of the arrangement of the fluorochromes in the probe (nucleotide) sequence, flouresce only when they are bound, so that the background is suppressed by unbound probes in cells, are produced.
(d) The probes are coupled to non-fluorescent/non-optical marker molecules which make it possible to detect the labeling by means of non-optical detection methods such as, for example atomic force microscopy or magnetic resonance.
(e) The probes are coupled to molecules which makes subsequent labeling possible as described in (a) to (d).
(f) The probes are constructed so that they have a suitable intrinsic fluorescence because of their chemical properties, or can be detected because of other specific properties even without modification.

The probe mixture is injected into the cell comprising the genome G by microinjection. It is also equally possible to employ all other known methods for introducing oligonucleotides into live cells.

After an incubation time whose duration in the case of live, proliferating cells is in proportion to the cell cycle times known to a skilled person, the relevant cell is examined with the aid of the detection system which is suitable for the chosen marker molecules—in the case of fluorescent dyes, subsequently with the aid of a fluorescence microscope—and the marker signals are detected:

Localization of the target DNA “T” of length L can take place for example by (a) all probes being labeled with fluorochromes of the same spectral signature S₁ for the plurality, where appropriate, of different partial sequences; or by b) one group of probes being labeled with a spectral signature S₁ and another group being labeled with a spectral signature S₂, or further parts being labeled with spectral signatures Sₙ; or by c) carrying out a combination of a) and b).

In case a), the discrimination (differentiation) of the probes bound to the target DNA “T” from nonspecifically bound probes takes place on the basis of the increased intensity of the fluorescence signal: since it is presumed that the diameter d_{T} of the target DNA is smaller than the width at half the chief maximum of the effective pixel function FWHM (or resolution-equivalent quantity of the detection system), the fluorescence emission intensities of the specifically bound probes in the region of the target DNA add up to a total intensity, while the nonspecifically bound probes are randomly distributed spatially in the object, with their average distance under suitable conditions being greater than the FWHM. As a consequence, the intensity of these isolated fluorescence signals (“background”) is considerably lower. If, for example, 10 probes are specifically bound to genome region T, and the other probes are randomly distributed in the specimen, then the location of genome region T can be identified on the basis of its fluorescence signal having an intensity which is about 10 times as strong.

In case b), the probes specifically bound to the target DNA “T” are identified on the basis of the colocalization of fluorescence signals differing in spectral signature. For example, T comprises only 3 binding sites for probes t₁, t₂, t₃, which have been labeled respectively with spectral signatures S₁, S₂ and S₃. In this case, the intensity of the fluorescence signals detected from the individual probes t₁, t₂ and t₃ at location T will not differ from the intensity of the “background signals”. The location T is, however, characterized by the simultaneous occurrence of fluorescence signals with spectral signatures S₁, S₂ and S₃ (where appropriate after correction of chromatic shifts occurring=spectral colocalization).

Case c) is a combination of the two embodiments a) and b): detection of the location of T takes place on the basis of a fluorescence signal which is stronger by comparison with the background for fluorochromes of a particular spectral signature and additionally on the basis of the spectral colocalization of two or more spectral signatures.

With simultaneous imaging of marker signals and genome, the presence, the intensity and, where appropriate, the simultaneous occurrence of the different marker signals at a particular location indicates where the target DNA or the gene X is localized in this genome.

### EXAMPLE 4

### Localization of a Plurality of Different Genes in the Same Genome

Two different genes, meaning two different target DNAs T₁, T₂ with respective lengths L₁, L₂ can be distinguished as follows:

a) The probe sequences specific for T₁ are all labeled with the same spectral signature S₁; the probes specific for T₂ are all labeled with a spectral signature S₂. The location of T₁ will be detected on the basis of the stronger (increased) fluorescence signal of spectral signature S₁; the location of T₂ will be detected on the basis of the stronger (increased) fluorescence signal of spectral signature S₂.
b) The probes specific for T₁ are labeled with fluorochromes of spectral signatures S₁, S₂, S₃; the probes specific for T₂ are labeled with fluorochromes of spectral signatures S₄, S₅, S₆. The location of T₁ is in this case detected by the spectral colocalization of S₁/S₂, S₃; the location of T₂ is detected by the spectral colocalization of S₄, S₅, S₆.
c) Procedures a) and b) are combined together, it being possible to vary the number and combination of the spectral signatures suitably. It will be appreciated by the skilled person that these procedures a) to c) can be carried out analogously also with more than two target DNAs, e.g. with the aid of an appropriate expansion of the number of signatures.

### EXAMPLE 5

### Localization of the Gene FMR1 on Human Chromosome X

Alterations in the gene FMR1 lead to the so-called fragile X syndrome which represents one of the commonest causes of hereditary cognitive impairment.

The nucleotide sequence of the FMR1 gene is known in the art and is available to the skilled person for example through a database (e.g. NCBI, ensemble, inter alia). This nucleotide sequence of the FMR1 gene is the target DNA in the present exemplary embodiment.

This target DNA was analyzed in relation to those partial sequences representing a unique pattern within the FMR1 gene. For the partial sequences found, the single-stranded probe sequences listed in table 1, which coincide with or are complementary to the partial sequences were provided.

All these probe sequences hybridize by Watson-Crick binding with one or another DNA single strand of the target DNA as soon as this DNA segment of the X chromosome is in the form of two single strands—which naturally occurs in vivo at least from time to time; for example during cell division and in this connection in particular during DNA duplication.

To carry out the localization of the FMR1 gene, a skilled person can if required stimulate or induce cell division of the patient's cell to be investigated and thus bring about the natural temporary splitting of the DNA into two DNA single strands.

Two or more of these probe sequences having a purine:pyrimidine ratio or pyrimidine:purine ratio of at least 3:16 are combined to give a probe set. The number of single probes used depends in this case on the sensitivity of detection of the microscope used. It is necessary to use more probes with a microscope of low sensitivity than with a microscope of high sensitivity, because the individual probes typically have only one or two fluorochromes.

It is also possible in principle for other probe sets to be produced for these genome regions defined in accordance with other parameters according to the invention.

The probe sequences of the probe set are coupled to fluorescent molecules (e.g. Oregon Green) of the same color signature, which are excited with laser light sources (e.g. at 488 nm) and their fluorescence is detected microscopically by a CCD camera. Each unit of probe sequence and marker molecule(s) exhibits virtually the same binding behavior and, under the given experimental conditions, an approximately identical melting point with the single strand, complementary thereto, of the target DNA as each of the other units.

The probe sequences are introduced into the cell of a patient's sample in vitro by microinjection and exposed to known hybridization conditions, for example incubation at 37° C. under standard cell culture conditions for 26 hours. After the hybridization phase has elapsed, the cells are fixed and the marker signals are detected in the cell nuclei. The location of the target DNA on the X chromosome is identified on the basis of the radial distance from the midpoint of the cell and by comparison with data from reference experiments with fixed cells and X-chromosomal standard FISH probes (FISH=fluorescence in situ hybridization). As supplement, it is possible to measure the DNA compaction by SMI microscopy (SMI=spatially modulated illumination) and draw conclusions from these measured results about the genetic condition of the labeling region.

### EXAMPLE 6

### Use of the Method of the Invention in Medical Diagnosis

The gene “RyR2” on chromosomes 1 of the human genome is associated with two diseases in which there is a balanced translocation between chromosome 1 and chromosome 14, namely (a) catecholaminergic polymorphic ventricular tachycardia and (b) right-ventricular dysplasia of type 2.

The breakpoint for this translocation is located within the RyR2 gene. However, it must be determined accurately in the patients, because its location may vary between individuals and therefore transcription of the defective gene may be started or not with the translocation.

In order to be able to delimit the location of the breakpoint on the gene, firstly the RyR2 gene, which represents the target DNA in this case, is analyzed with the aid of known genome databases in relation to a plurality of partial sequences having a purine:pyrimidine ratio of 0:N (with N=14, 15, . . . to 21). Subsequently, a probe set is provided of 16 individual probes which are coincident with or complementary to the partial sequences of the target DNA. All 16 probes hybridize by Watson-Crick binding with complementary sequences along the RyR2 gene, with the individual probes being distributed over the entire length of the gene after hybridization has taken place. The composition of this probe set is depicted in table 2.

The probe sequences are coupled at each of their ends to fluorescent molecules of the same color signature. In this case, two aliquots differing in their color signature, e.g. Oregon Green (green) and TAMRA (red), are prepared for each probe sequence.

To determine the location of the breakpoint, 16 variants of the probe set differing in the color labeling of the probes in such a way that in each variant the proportion of red-labeled probes increases by one probe, and the proportion of green-labeled probes decreases by one probe, are provided. In this case the probes are labeled initially with red and later with green in the sequence of their arrangement on the target DNA. Sixteen probe set variants result, SS₁ to SS₁₆, where probe set variant SS₁ comprises probe S₁ with a green label and probes S₂ to S₁₆ with a red label, probe set variant SS₂ comprises probes S₁ and S₂ with a green label and probes S₃ to S₁₆ with a red label, probe set variant SS₃ comprises probes S₁, S₂ and S₃ with a green label and probes S₄ to S₁₆ with a red label etc. (see FIG. 1).

A hybridization procedure with the target DNA is carried out with each of these 16 probe set variants. For this purpose, the probe sets are applied to fixed cells of a patient's sample and exposed to standard hybridization conditions.

After the hybridization phase has elapsed, the marker signals in the cell nuclei are detected.

The probe set variant which provides two single-colored marker signals shows the location of the breakpoint because the breakpoint is located in the region between the two probe regions Sₙ and Sₙ₊₁ of the probe set variant SSₙ which provides a single-colored marker signal (green or red) after the hybridization (see FIG. 1).

In order to determine the location of the breakpoint even more accurately, a new probe set SS* whose individual probes have a purine-pyrimidine ratio of 1:N (with N=14, 15, . . . to 21) is produced for the region between the two probe regions Sₙ and Sₙ₊₁ found.

Variants SS*₁ to SS*ₙ of this probe set SS* which differ in the color labeling (for example red or green) of their individual probes, in analogy to the probe set variants SS₁ to SS₁₆, are again provided.

A hybridization procedure is carried out with the target DNA with each of these n probe set variants, the probe sets again being applied to fixed cells of a patient's sample and exposed to standard hybridization conditions.

After the hybridization phase has elapsed, the marker signals are again detected in the cell nuclei.

The probe set variant which provides two single-colored marker signals again indicates the location of the breakpoint between probe region S*ₙ and S*n+1 of the probe set variant SS*ₙ.

### EXAMPLE 7

### Use of the Method of the Invention in Pathology

The increased number of copies of the genes “Her2neu” and “GRB7” on chromosome 17 of the human genome is of substantial importance for a therapeutic decision in cases of ductal breast cancer. The two genes have a length of about 30 kb (Her2neu) and about 10 kb (GRB7) and are only about 10 kb apart. Conventional, generally available probes from modified BAC clones are therefore unable to demonstrate these genes separately.

For microscopic diagnosis according to the invention on tissue sections, a probe set each composed of 18 individual probes was produced for each of these two genes, and the total number of nucleotides in all the individual probes of each probe set was to be 300 or more and the difference in the total number of nucleotides between the two probe sets was not to exceed 5%. A specific example of a very suitable composition of these two probe sets is depicted in table 3 and table 4.

The individual probes of each set are labeled with in each case two fluorescent molecules of the same color signature for the microscopic analysis. The two probe sets comprise different color signatures. The hybridization takes place in accordance with standard protocols because the probes are capable of Watson-Crick bindings.

The microscopic analysis takes place on the basis of images recorded by a CCD camera in an epifluorescence microscope. Finally, the number of labels (color points) is counted and the number of copies of the relevant gene is deduced therefrom. The found number provides the clinician with an indication of the suitable therapy.

### EXAMPLE 8

### Further Possible Uses

The method of the invention can also be carried out with investigation material fixed in any way. This is associated in particular with the advantages that, with a suitable choice of probes, a denaturation step may be dispensed with, and that the probe mixture can be manipulated just as simply as, for example, established DNA stains in clinical cytogenetics:

The probe mixture is added to the specimen, incubated, possibly briefly washed and finally evaluated microscopically.

A further advantage over known FISH methods at room temperature is that there is no need to employ chaotropic chemical agents which have a toxic effect and which may cause allergic reactions when handled.

In sensor technology it is possible to construct appropriate DNA chips which recognize target DNAs on addition onto given probe sequences, without the need to subject the chip or the investigation material additionally to a chemical and/or thermal treatment. The requirements for the optical system used to evaluate these DNA chips can also be considerably reduced. For example, it is possible to employ microscope optics of lower numerical aperture as long as the dimensions of the chip are appropriately adapted and, for example, nanocrystals are used. Since very complex systems are now available for the optical analysis of DNA chips, the method can be made considerably more economic in this way.

The method of the invention has proved to be very applicable in practice to the following genes:

NRAS[1], AKT3[1], CDC2L1(p58)[1], CDC2L2(p58)[1] ABL2[1], RYR2[1], MSH2[2], GNLY[2], RASSF1[3], FHIT[3], EGF[4], ENC1[5], MCC[5], HMMR[5], ECG2[5], PIM1[6], ABCB1 (MDR)[7], MET[7], (C-)MYC[8], CDKN2A[9], ABL1[9], PTEN[10], PGR[11], ATM[11], KRAS2[12], RB1[13], PNN[14], IGH[14], SNRPN[15], IGF1R[15], UBE3A[15], PML[15], FANCA[16], CDH13[16], D17S125[17], ERBB2 (HER2neu)[17], TP53 (p53)[17], PSM3(p58subunit)[17], RARA[17], GRB7[17], LAMA3[18], AKT2[19], TNFRSF6B[20], MYBL2[20], PTPN1[20], ZNF217[20], PCNT2[21], TFF1-3[21], PDGFB (SIS)[22], TBX1[22], BCR[22], ERAS[X], PIM2[X], RAB9A[X], DAZ4[Y], DAZ3[Y].

TABLE 1

Individual probes of the probe set in example 5
(for localization of the FMR1 gene of the human X
chromosome)

| | |
|---|---|
| (1) | AACCTTTCTTTTCTCTTCCAA |
| (2) | CCAAAAGAAGAAAGAAGGTC |
| (3) | TTAAAGAGAGGAGAAGGTG |
| (4) | GTGAGAGAGAAAGAGAAAGAGAGTG |
| (5) | TATTCTTTCCTTTTCTTTTAC |
| (6) | TTGAGAGGGAAAGGGAATA |
| (7) | ATGGGAGAGGAGGAGAAGATG |
| (8) | AGTTCCTTTTCCTTTCCAT |
| (9) | GATCCTTCCTTTCTCCCCTGT |
| (10) | CTGAAGGAAGGAAGGAGGGAGGAAGGAAGGAAGCA |
| (11) | GTAGAGGGAAGGGAGAAAGGTG |
| (12) | TCAAAAGAAGGAGAAGATG |
| (13) | TGTCTTCCTTTCTTCCTCCAT |
| (14) | TGTCTCTCTCTCTCCTCCCCCCC |
| (15) | CACCCCTCTCTCTCCTTCTCTCTTTTCTGT |
| (16) | TGCTCCCCTCCCTCCTCAC |

TABLE 2

Individual probes of the probe set SS in example 6
(for localization of the breakpoint on the RyR2
gene of human chromosome 1)

| | |
|---|---|
| (1) | CCTCCTCTTCTCTCCCTCCC |
| (2) | TCTTCCTTTTCTCTCT |
| (3) | CTTTCCCTCCCTCTT |
| (4) | CCTTCTTCTTTCCCC |
| (5) | CCTCCTCTTTTCTCCT |
| (6) | TTCCTTCCTTCCCTCTTTCC |
| (7) | CTCTTTTCTTTTCTTTTCTTTCTT |
| (8) | CTCCTTCCTTTCTCTC |
| (9) | TTCTCTTTCTTCTCTTCT |
| (10) | TTTCTCTTTCCTCCT |
| (11) | TCTTTCCCTTTCCCTCC |
| (12) | CCTCCCTTCCCCCTTCC |
| (13) | CTCCTTTTCCCTTCCCT |
| (14) | CCCTTTCCCTTTCTCTCCC |
| (15) | CCCCCTTCCTCCCCTTTC |
| (16) | TCTCTCTCCCTCCCCTC |
| (17) | TCTTCTCTCCTCTTCTCTCCTC |

TABLE 3

Individual probes of the probe set for GRB7 in
example 7

| | |
|---|---|
| (1) | CTTCCTCCCTTCTCCTCC |
| (2) | CCCTCCCCCCTCCCTCCC |
| (3) | CCCCTTCCTCCTCCCT |
| (4) | CCTCCTTCTCCCCTCT |
| (5) | CCTCTCTCCCTTTTTCTTCTT |
| (6) | AGAAGGGAAGGGAGGGA |
| (7) | AGAGGGGAAGGGAGGAGG |
| (8) | CTCTCTTTCTCTCCCC |
| (9) | CCTCTCTCCTCTCCTTCC |
| (10) | AGGAGGGAGGAAGAGAGGG |
| (11) | CCCCCTCTCCTTCTCCT |
| (12) | GGGGAGGAGAGAAAAGAAGGAG |
| (13) | CCTCCCTTTCCTCTCCC |
| (14) | AGGAGAGGAGAGAGAGGGAAGA |
| (15) | CCTTCCTTCCCCCCTCTCCCCT |
| (16) | GGGGGAGGGGAAGAAGGAGGG |
| (17) | TTCCTTTCTCCTCCTC |
| (18) | CTCCCTTTTCTCTTCTT |

TABLE 4

Individual probes of the probe set for Her2neu in
example 7

| | |
|---|---|
| (1) | CTTTCTCCCCTTCTCCCTC |
| (2) | AAGGAGAAAAGGAGGA |
| (3) | GAGGGGAGAAGGGAGG |
| (4) | CCTCCTCTCTCTCCC |
| (5) | GGGAAGGAGAAGAGGAAGG |
| (6) | CCCCTCCTCCTTCCTCT |
| (7) | AGAGGAAGAGAAGAA |
| (8) | CCTTCTTTCCTCTCTCCTTCCC |
| (9) | CCCTTTCTCCTCCCCC |
| (10) | TCTCTTTTCCTTTCTCTTCCCCCTCCTC |
| (11) | AAAGGAAGAGAAGAA |
| (12) | CCCCTTTCCCTTCCCT |
| (13) | CCCCCCTTCCTCTCCTCTT |
| (14) | GGGGGGGAGGGAAGAGAGAAAGAGA |
| (15) | GAAGAGAGGGAGAAAG |
| (16) | GAGAAGGAAGGAGAGAG |
| (17) | CCTCTTTCCTCCTCTC |
| (18) | TCCTTCCCTCCCCCTCT |

1. A method for microscopic analysis of a localization of a selected intracellular native genome segment having a known nucleotide sequence target DNA within a genome in situ, wherein
(1) the target DNA is analyzed based on genome databases for
(a) a single or
(b) a plurality of identical or different partial sequences,
wherein these partial sequences or a combination of the plurality of identical or different partial sequences represent a unique pattern within the genome,
(2) single-stranded probe sequences are provided which correspond to or are complementary to the partial sequence(s) of the target DNA,
and which are suitable for hybridization via a Watson-Crick binding to the partial sequence(s) which is/are, naturally at least temporarily, in form of two DNA single strands of the target DNA,
(3) said probe sequences are coupled to identical and/or different marker molecules which can be detected by microscopy or spectroscopy or by magnetic resonance, and
wherein in case (1b) of a combination of a plurality of partial sequences or probe sequences, respectively, and coupling thereof to different marker molecule(s), each unit of probe sequence and marker molecule(s) displays virtually identical binding behavior or an identical melting point with the single strand of the target DNA complementary thereto as another of said units,
(4) said probe sequences are introduced into the cell and brought together with the target DNA by known methods in such a way that they hybridize with the corresponding partial sequence(s) of the target DNA, which is/are temporarily in form of two DNA single strands,
(5) emitted marker signals are detected, and
(6) location of the target DNA on the genome is identified on based on presence and/or the intensity and/or the simultaneous occurrence of different marker signals.

2. A method according to claim 1, wherein the method is carried out in vivo.

3. A method according to claim 1 or 2, wherein each partial sequence includes at least 8 nucleotides.

4. A method according to claim 1, wherein the marker molecules are fluorescent dyes and/or immunohistochemical marker molecules, and/or fluorescent or luminescent or magnetic nanoparticles.

5. A method according claim 1, wherein the method localizes and detects point mutations and/or genome breakpoints and/or equivalent sequence defects wherein:
in (1) the target DNA is analyzed for a plurality of identical or different partial sequences t₁-tₙ,
in (3) probe sequences S₁-Sₙ are coupled to different marker molecules which can be detected by microscopy or spectroscopy or by magnetic resonance, and
in (6) the absence of the marker signals of one or more probe sequences Sₓ with simultaneous presence of the marker signals of probe sequences Sₙ-Sₓ indicates presence of a point mutation or a genome breakpoint or an equivalent sequence defect.

6. A method according to claim 1, wherein the probe sequences are introduced into the cell by microinjection.

7. A probe set for in situ hybridization and microscopic analysis comprising
a single or a plurality of probe type(s) S₁-Sₙ, wherein each probe type Sᵢ comprises/consists of an oligonucleotide or an oligonucleotide-equivalent molecule which is complementary or identical to a single-stranded partial sequence tᵢ of an intracellular, double-stranded genome segment having a known nucleotide sequence target DNA wherein
the single or the plurality of partial sequences in combination represent a unique pattern within the genome,
the probes are suitable for hybridization via Watson-Crick binding to the partial sequence(s) which is/are, naturally at least temporarily, in form of two DNA single strands of the target DNA,
and
the probes are coupled to identical and/or different marker molecules which can be detected by microscopy or spectroscopy or by magnetic resonance, wherein in the case of combination of a plurality of probe types and their coupling to different marker molecule(s) each unit of probe type and marker molecule(s) shows virtually identical binding behavior or an identical melting point with the single strand of the target DNA complementary thereto as another of said units.

8. A probe set according to claim 7, wherein the oligonucleotide or the oligonucleotide-equivalent molecule includes at least 8 nucleotides.

9. A probe set according to claim 7 or 8, wherein the marker molecules are fluorescent dyes and/or immunohistochemical marker molecules, and/or fluorescent or luminescent or magnetic nanoparticles.

10. A method according to claim 3, wherein each partial sequence includes 10-40 nucleotides.

11. A method according to claim 4, wherein the nanoparticles are nanocrystals (quantum dots).

12. A probe set according to claim 8, wherein the oligonucleotide or the oligonucleotide-equivalent molecule includes 10-40 nucleotides.

13. A probe set according to claim 9, wherein the nanoparticles are nanocrystals (quantum dots).
